(19) United States
(12) Patent Application Publication
(10) Pub. No.: US 2026/0306104 A1
(43) Pub. Date: Oct. 8, 2026

(54) OCCLUSIVE IMPLANT SYSTEM

(71) Applicant: BOSTON SCIENTIFIC SCIMED, INC., MAPLE GROVE, MN (US)

(72) Inventors: Neal Thomas Eidenschink, Wayzata, MN (US); Chris Clark, Maple Grove, MN (US); Daniel L. Ross, Watertown, MN (US); Thyna M. Chau, Rochester, MN (US); Ted William Pelzer, Otsego, MN (US); Harishankar Natesan, Minneapolis, MN (US); Mark Christian McPhail, Maple Grove, MN (US)

(73) Assignee: BOSTON SCIENTIFIC SCIMED, INC., MAPLE GROVE, MN (US)

(21) Appl. No.: 19/636,231

(22) Filed: Apr. 1, 2026

Related U.S. Application Data
(60) Provisional application No. 63/782,368, filed on Apr. 2, 2025.

(51) Int. Cl. A61B 17/12; A61B 17/00

(52) U.S. Cl. CPC A61B 17/12172; A61B 2017/00477

(57) ABSTRACT

An occlusive implant system includes an occlusive implant including an expandable framework including a proximal hub, a perimeter shoulder portion spaced apart radially outward from the proximal hub, and a proximal face, and an occlusive covering secured to the framework. The covering extends from the proximal hub, across the proximal face, and over the perimeter shoulder portion. The covering is mechanically secured to the proximal face at a plurality of locations. The covering may extend over the perimeter shoulder portion to a plurality of side attachment points disposed along a laterally facing surface of the framework. The covering may be secured directly to the proximal face at the plurality of locations. In a deployed configuration, each of the plurality of locations is disposed distal of the perimeter shoulder portion.

### CROSS-REFERENCE TO RELATED APPLICATIONS

This application claims the benefit of priority of U.S. Provisional Application No. 63/782,368 filed Apr. 2, 2025, the entire disclosure of which is hereby incorporated by reference.

### TECHNICAL FIELD

The disclosure relates generally to medical devices and more particularly to an occlusive implant system including an occlusive implant and an occlusive covering.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Over time, exposed surface(s) of an implant spanning the left atrial appendage may become covered with tissue (a process called endothelization), effectively removing the left atrial appendage from the circulatory system and reducing or eliminating the thrombi which may enter the blood stream from the left atrial appendage.

In some instances, the tissue covering the exposed surface(s) of the implant may be misdiagnosed during follow up examinations as a procedural complication known as device-related thrombus (DRT). DRT is sometimes identified via hypoattenuation thickening (HAT) using cardiac computed tomography (CT) imaging. Of the known medical devices and methods, each has certain advantages and disadvantages. There is an ongoing need to provide alternative medical devices, as well as alternative methods for manufacturing and using medical devices, which may reduce the possibility of misdiagnosed DRT.

### SUMMARY

In one example, an occlusive implant system may comprise an occlusive implant comprising an expandable framework configured to shift between a collapsed configuration and a deployed configuration, wherein the expandable framework comprises a proximal hub, a perimeter shoulder portion spaced apart radially outward from the proximal hub in the deployed configuration, and a proximal face extending between the proximal hub and the perimeter shoulder portion, and an occlusive covering secured to the expandable framework. The occlusive covering may extend from the proximal hub, across the proximal face of the expandable framework, and over the perimeter shoulder portion. The occlusive covering may be mechanically secured to the proximal face of the expandable framework at a plurality of locations.

In addition, or alternatively, to any example disclosed herein, the proximal face of the expandable framework includes a distally recessed portion surrounding the proximal hub in the deployed configuration.

In addition, or alternatively, to any example disclosed herein, the proximal face of the expandable framework includes a proximal surface disposed radially outward of the distally recessed portion and proximal of the distally recessed portion in the deployed configuration.

In addition, or alternatively, to any example disclosed herein, at least a portion of the occlusive covering extending across the proximal face of the expandable framework extends distally of the proximal surface.

In addition, or alternatively, to any example disclosed herein, at least a portion of the occlusive covering is disposed within the distally recessed portion.

In addition, or alternatively, to any example disclosed herein, the plurality of locations is disposed within the distally recessed portion.

In addition, or alternatively, to any example disclosed herein, the occlusive covering is sutured to the proximal face of the expandable framework.

In addition, or alternatively, to any example disclosed herein, the occlusive covering is mechanically secured to the proximal face of the expandable framework with a plurality of melted plugs.

In addition, or alternatively, to any example disclosed herein, the occlusive covering is mechanically secured to the proximal face of the expandable framework with a plurality of hooks monolithically formed with the expandable framework.

In addition, or alternatively, to any example disclosed herein, the occlusive covering is mechanically secured to the proximal face of the expandable framework with a wire extending circumferentially around the proximal hub.

In addition, or alternatively, to any example disclosed herein, the wire is interwoven through the occlusive covering and the proximal face of the expandable framework.

In addition, or alternatively, to any example disclosed herein, a plurality of first segments of the wire is disposed proximal of the occlusive covering and a plurality of second segments of the wire is disposed distal of the proximal face of the expandable framework.

In addition, or alternatively, to any example disclosed herein, and in a second example, an occlusive implant system may comprise an occlusive implant comprising an expandable framework configured to shift between a collapsed configuration and a deployed configuration, wherein the expandable framework comprises a proximal hub, a perimeter shoulder portion spaced apart radially outward from the proximal hub in the deployed configuration, and a proximal face extending from the proximal hub to the perimeter shoulder portion, and an occlusive covering secured to the proximal hub and the expandable framework. The occlusive covering may extend from the proximal hub, across the proximal face of the expandable framework, and over the perimeter shoulder portion to a plurality of side attachment points disposed along a laterally facing surface of the expandable framework. The occlusive covering may be secured directly to the proximal face of the expandable framework at a plurality of locations.

In addition, or alternatively, to any example disclosed herein, the expandable framework comprises a plurality of interconnected struts.

In addition, or alternatively, to any example disclosed herein, the occlusive covering is secured directly to the plurality of interconnected struts at the plurality of locations within a distally recessed portion of the proximal face.

In addition, or alternatively, to any example disclosed herein, the plurality of interconnected struts comprises apertures formed within individual struts of the plurality of interconnected struts at the plurality of locations.

In addition, or alternatively, to any example disclosed herein, the plurality of interconnected struts comprises lateral notches formed within individual struts of the plurality of interconnected struts at the plurality of locations.

In addition, or alternatively, to any example disclosed herein, in the deployed configuration, the occlusive covering extends along a path that extends distally from the proximal hub to the plurality of locations and proximally from the plurality of locations toward the perimeter shoulder portion.

In addition, or alternatively, to any example disclosed herein, the plurality of locations is circumferentially spaced apart equidistantly around the proximal hub.

In addition, or alternatively, to any example disclosed herein, and in a third example, an occlusive implant system may comprise an occlusive implant comprising an expandable framework configured to shift between a collapsed configuration and a deployed configuration, wherein the expandable framework comprises a proximal hub, a perimeter shoulder portion spaced apart radially outward from the proximal hub in the deployed configuration, and a proximal face extending between the proximal hub and the perimeter shoulder portion, and an occlusive covering extending radially outward from the proximal hub across the proximal face of the expandable framework. The occlusive covering may be mechanically secured to the proximal face of the expandable framework at a plurality of locations. In the deployed configuration, each of the plurality of locations may be disposed distal of the perimeter shoulder portion.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-2 illustrate selected aspects of an occlusive implant system comprising an occlusive implant for occluding a left atrial appendage;

FIG. 3 illustrates selected aspects of an occlusive implant including an occlusive covering secured thereto;

FIG. 4 illustrates selected aspects of the occlusive implant;

FIG. 5 is a cross-sectional view illustrating selected aspects of the occlusive implant including the occlusive covering secured thereto;

FIGS. 6A-6B illustrate selected aspects of an alternative configuration of the occlusive implant;

FIGS. 7A-7B illustrate selected aspects of an alternative configuration of the occlusive implant;

FIGS. 8A-8B illustrate selected aspects of an alternative configuration of the occlusive implant;

FIGS. 9A-9B illustrate selected aspects of an alternative configuration of the occlusive implant;

FIGS. 10A-10B illustrate selected aspects of an alternative configuration of the occlusive implant; and

FIGS. 11-12 illustrate selected aspects of an alternative configuration of the occlusive implant.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

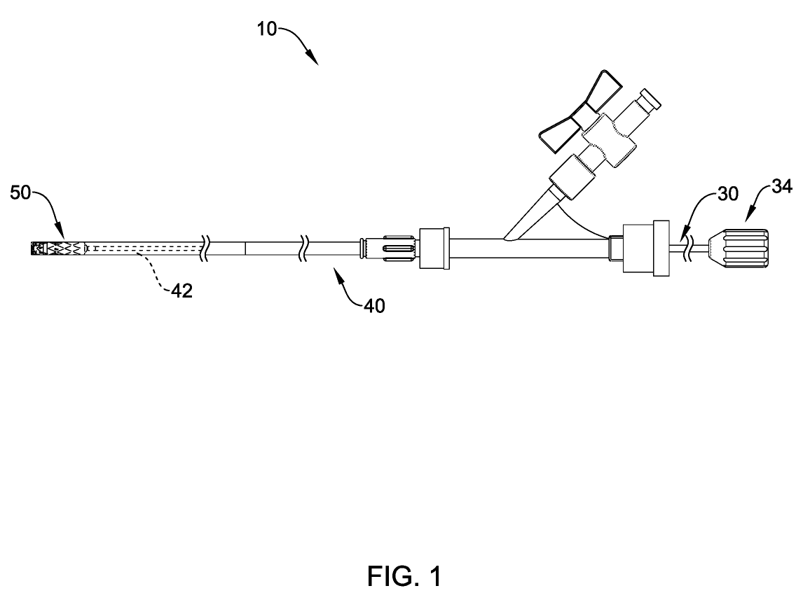

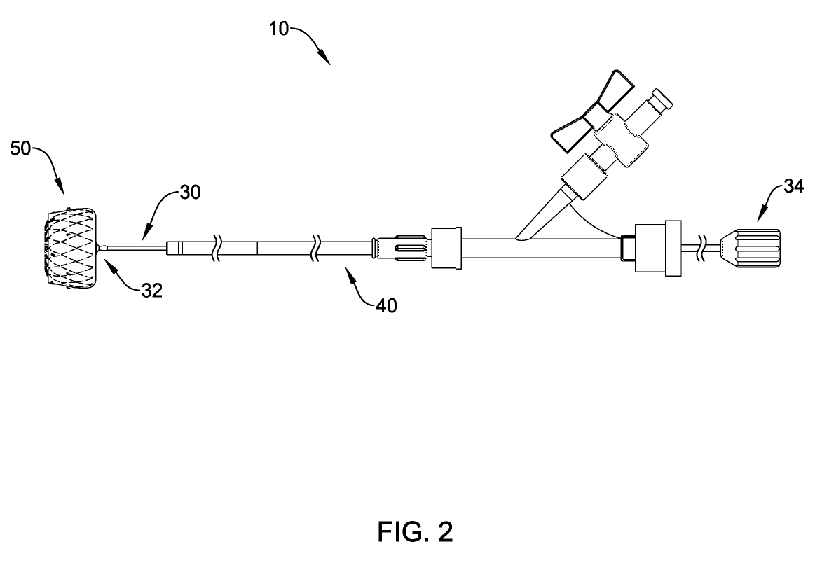

FIGS. 1-2 illustrate selected components and/or arrangements of an occlusive implant system 10 for occluding a left atrial appendage. It should be noted that in any given figure, some features of the occlusive implant system 10 may not be shown, or may be shown schematically, for simplicity. Additional details regarding some of the components of the occlusive implant system 10 may be illustrated in other figures in greater detail. The occlusive implant system 10 may be used to percutaneously deliver and/or deploy a variety of medical implants (e.g., a cardiovascular implant, an occlusive implant, a replacement heart valve implant, etc.) to one or more locations within the anatomy, including but not limited to, in some embodiments, the heart.

The occlusive implant system 10 may comprise a core wire 30. The occlusive implant system 10 may comprise an occlusive implant 50 releasably couplable to and/or disposable at a distal end 32 of the core wire 30. In at least some embodiments, the occlusive implant 50 may be configured to occlude the left atrial appendage. The left atrial appendage is attached to and in fluid communication with the left atrium of the patient’s heart. The left atrial appendage may have a complex geometry and/or irregular surface area. In some patients, the left atrial appendage may have a plurality of lobes and/or recesses extending in different directions.

In some embodiments, the occlusive implant system 10 may include a delivery sheath 40 having a lumen 42 (e.g., FIG. 1) extending from a proximal opening to a distal opening. In some embodiments, the core wire 30 may be slidably disposed within the lumen 42 of the delivery sheath 40. The core wire 30 may have a proximal end 34 disposed proximal of the delivery sheath 40. In some embodiments, the proximal end 34 of the core wire 30 may include a knob and/or a handle configured to manipulate and/or move the core wire 30 and/or the occlusive implant 50. In some embodiments, the proximal end 34 of the core wire 30 may include a knob and/or a handle configured to manipulate and/or move the core wire 30 and/or the occlusive implant 50 relative to the delivery sheath 40. In some embodiments, the delivery sheath 40 may be sized and configured to deliver the occlusive implant 50 to the left atrial appendage.

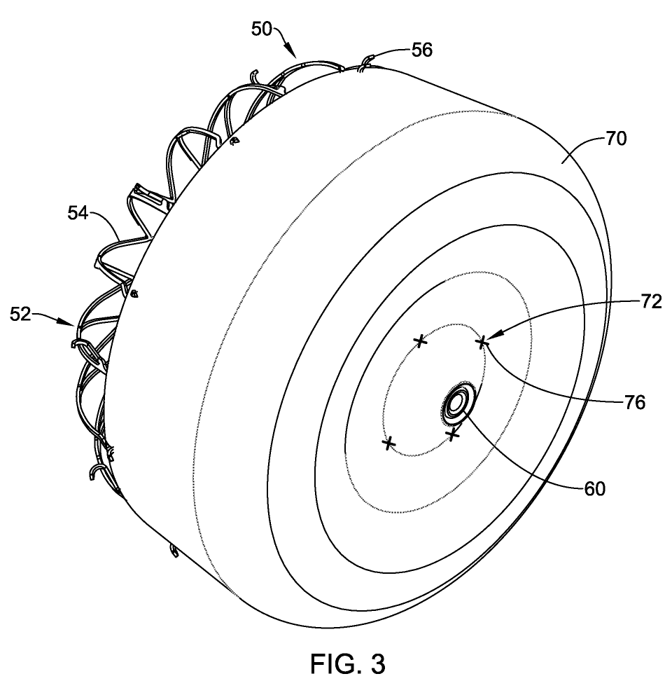

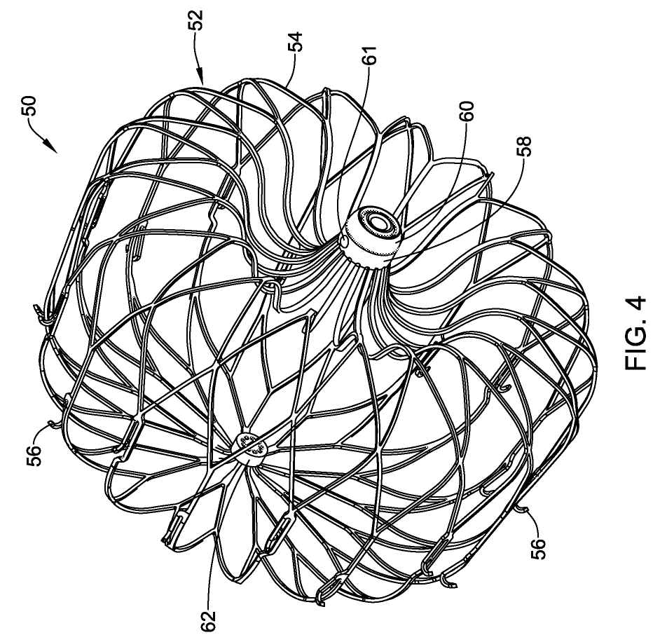

The occlusive implant 50 may include an expandable framework 52 (e.g., FIGS. 3-4) configured to shift between a collapsed configuration (e.g., FIG. 1) and a deployed configuration (e.g., FIGS. 2-4). In some embodiments, the occlusive implant 50 may be disposed within the lumen 42 of the delivery sheath 40 proximate the distal opening in the collapsed configuration. In some embodiments, the delivery sheath 40 may constrain the occlusive implant 50 and/or the expandable framework 52 in the collapsed configuration. In some embodiments, the occlusive implant 50 and/or the expandable framework 52 may be configured to shift from the collapsed configuration toward and/or to the deployed configuration when the occlusive implant 50 is disposed distal of the distal opening of the lumen 42 and/or the delivery sheath 40, and/or when the occlusive implant 50 is unconstrained. In some embodiments, the occlusive implant 50 and/or the expandable framework 52 may be configured to shift from the collapsed configuration toward and/or to the deployed configuration when the occlusive implant 50 is unconstrained by the delivery sheath 40. In at least some embodiments, the expandable framework 52 may be self-biased toward the deployed configuration.

In some embodiments, the core wire 30 may be slidably and/or rotatably disposed within the lumen 42 of the delivery sheath 40. In some embodiments, the proximal end 34 of the core wire 30 may extend proximally of a proximal end of the delivery sheath 40 and/or the proximal opening of the lumen 42 for manual manipulation by a clinician or practitioner. In some embodiments, the occlusive implant 50 may be removably attachable, joinable, securable, or otherwise connectable to the distal end 32 of the core wire 30. The core wire 30 may be configured to and/or may be capable of axially translating the occlusive implant 50 relative to the delivery sheath 40. The delivery sheath 40 and/or the core wire 30 may have a selected level of axial stiffness and/or pushability characteristics while also having a selected level of flexibility to permit navigation through the patient’s vasculature.

Some suitable, but non-limiting, examples of materials for the occlusive implant system 10, the core wire 30, the delivery sheath 40, and/or the occlusive implant 50, etc. are discussed below.

Turning now to FIGS. 3-4, the occlusive implant 50 may comprise an expandable framework 52 configured to shift along a longitudinal axis between the collapsed configuration (e.g., FIG. 1) and the deployed configuration (e.g., FIGS. 2-4). In the collapsed configuration, the expandable framework 52 may be axially elongated and/or radially compressed. In the deployed configuration, the expandable framework 52 may be axially shortened and/or radially expanded. The expandable framework 52 may comprise a plurality of interconnected struts 54 defining a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements. In some embodiments, the plurality of interconnected struts 54 may converge, join, and/or connect at intersections or nodes.

The plurality of interconnected struts 54 may be formed and/or cut from a tubular member. In some embodiments, the plurality of interconnected struts 54 may be monolithically formed and/or cut from a monolithic member. In some embodiments, the plurality of interconnected struts 54 may be integrally formed and/or cut from a monolithic tubular member and subsequently formed and/or heat set to a desired shape in the deployed configuration. In some embodiments, the plurality of interconnected struts 54 may be monolithically formed and/or cut from a monolithic flat member or sheet, and then rolled or formed into a tubular structure and subsequently formed and/or heat set to the desired shape in the deployed configuration. Some exemplary means and/or methods of making and/or forming the plurality of interconnected struts 54 include laser cutting, machining, punching, stamping, electro discharge machining (EDM), chemical dissolution, etc. Other means and/or methods are also contemplated.

In some embodiments, the expandable framework 52 may be compliant and substantially conform to and/or be in sealing engagement with the shape and/or geometry of a wall of the left atrial appendage in the deployed configuration. In some embodiments, the occlusive implant 50 may expand to a size, extent, or shape less than or different from a maximum unconstrained extent, as determined by the surrounding tissue and/or wall of the left atrial appendage. In some embodiments, reducing a thickness of various elements of the expandable framework 52 may increase the flexibility and compliance of the expandable framework 52 and/or the occlusive implant 50, thereby permitting the expandable framework 52 and/or the occlusive implant 50 to conform to the tissue around it, rather than forcing the tissue to conform to the expandable framework 52 and/or the occlusive implant 50. In some embodiments, the expandable framework 52 and/or the occlusive implant 50 may be stronger and/or less compliant, and thus the expandable framework 52 and/or the occlusive implant 50 may force the tissue of the left atrial appendage to conform to the expandable framework 52 and/or the occlusive implant 50 in the deployed configuration. Other configurations are also contemplated.

In some embodiments, the occlusive implant 50 and/or the expandable framework 52 may comprise a plurality of anchoring elements 56. In some embodiments, the plurality of anchoring elements 56 may extend radially outward from the expandable framework 52 and/or the plurality of interconnected struts 54 in the deployed configuration. In at least some embodiments, the plurality of anchoring elements 56 may be configured to engage with tissue and/or may be configured to secure the occlusive implant 50 and/or the expandable framework 52 to tissue at a target site (e.g., the left atrial appendage, etc.). In some embodiments, the plurality of anchoring elements 56 may be configured to prevent dislodgement and/or ejection of the occlusive implant 50 from the target site and/or the left atrial appendage.

In some embodiments, the occlusive implant 50 and/or the expandable framework 52 may comprise a proximal collar 58, a proximal hub 60, and/or a distal hub 62. The longitudinal axis of the expandable framework 52 may extend from the proximal hub 60 to the distal hub 62. In at least some embodiments, the proximal hub 60 and/or the distal hub 62 may be centered on and/or coaxial with the longitudinal axis.

The plurality of interconnected struts 54 may extend between the proximal collar 58, and/or the proximal hub 60, and the distal hub 62. The plurality of interconnected struts 54 may be joined together at and/or fixedly attached to the proximal collar 58, and/or the proximal hub 60, and/or the distal hub 62. In some embodiments, the proximal collar 58 may be circumferentially continuous. In some embodiments, the proximal collar 58 may be monolithically formed with the plurality of interconnected struts 54. For example, proximal ends of the plurality of interconnected struts 54 may be monolithically formed with the proximal collar 58 and/or the plurality of interconnected struts 54 may extend distally from the proximal collar 58. In some embodiments, the proximal hub 60 may be secured within the proximal collar 58. In some embodiments, the proximal hub 60 may be fixedly attached to and/or fixedly secured within the proximal collar 58. In one non-limiting example, the proximal hub 60 may be secured within the proximal collar 58 by a pin member 61 extending laterally therethrough. Other configurations are also contemplated. In some embodiments, the distal hub 62 may be fixedly attached to the expandable framework 52 and/or distal ends of the plurality of interconnected struts 54, such as by welding, adhesive bonding, brazing, soldering, etc. Other configurations are also contemplated.

In some embodiments, the proximal hub 60 may be configured to releasably connect, couple, and/or attach the occlusive implant 50 and/or the expandable framework 52 to the distal end 32 of the core wire 30 (e.g., FIGS. 1-2). In some embodiments, the proximal hub 60 may include internal threads configured to rotatably and/or threadably engage external threads formed on and/or at the distal end 32 of the core wire 30. Other configurations and/or structures for releasably securing the occlusive implant 50 to the core wire 30, including but not limited to pins, springs, detents, etc., are also contemplated.

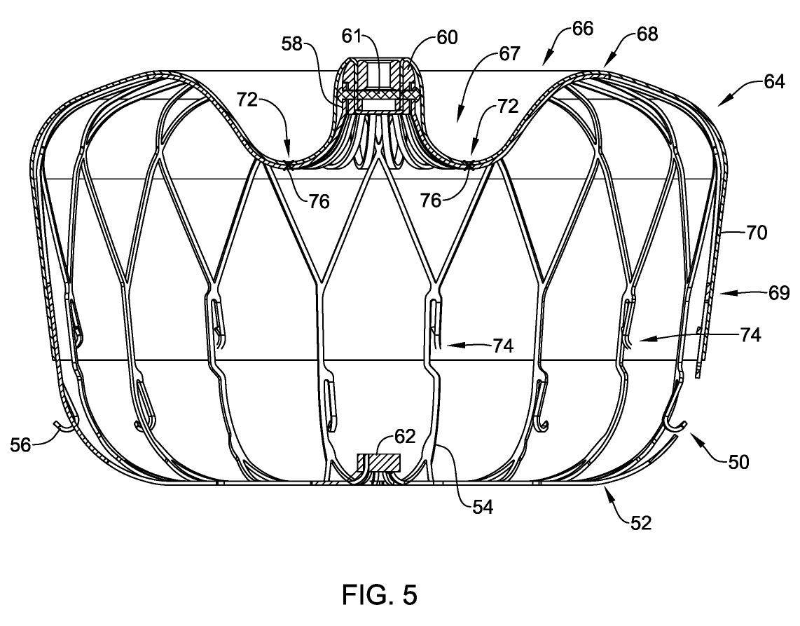

In some embodiments, the expandable framework 52 and/or the plurality of interconnected struts 54 may comprise a perimeter shoulder portion 64 disposed at a radially outward perimeter of the expandable framework in the deployed configuration, as seen in FIG. 5. In some embodiments, the perimeter shoulder portion 64 may be spaced apart radially outward from the proximal collar 58 and/or the proximal hub 60 in the deployed configuration. In some embodiments, the expandable framework 52 may comprise a proximal face 66 extending between the proximal collar 58 and/or the proximal hub 60 and the perimeter shoulder portion 64 in the deployed configuration.

In some embodiments, the proximal face 66 of the expandable framework 52 and/or the plurality of interconnected struts 54 may include a distally recessed portion 67 surrounding the proximal collar 58 and/or the proximal hub 60 in the deployed configuration. In some embodiments, the proximal face 66 of the expandable framework 52 and/or the plurality of interconnected struts 54 may include a proximal surface 68 disposed radially outward of the distally recessed portion 67 and/or radially between the distally recessed portion 67 and the perimeter shoulder portion 64 in the deployed configuration. In some embodiments, the proximal surface 68 of the proximal face 66 may be disposed proximal of the distally recessed portion 67 in the deployed configuration. In some embodiments, the proximal surface 68 of the proximal face 66 may be a proximalmost extent of the expandable framework 52 and/or the plurality of interconnected struts 54 between the proximal collar 58 and/or the proximal hub 60 and the perimeter shoulder portion 64 in the deployed configuration.

With reference to FIG. 3 and the cross-sectional view of FIG. 5, in some embodiments, the occlusive implant system 10 may comprise an occlusive covering 70 secured to, attached to, connected to, disposed on, and/or disposed over a proximal portion of the expandable framework 52 and/or the plurality of interconnected struts 54 of the occlusive implant 50. In some embodiments, the occlusive covering 70 may be secured to and/or attached to the proximal hub 60 and/or may be secured to and/or attached to the expandable framework 52 at the proximal hub 60. In some embodiments, the occlusive covering 70 may extend radially outward from and/or may extend distally from the proximal hub 60. In some embodiments, the occlusive covering 70 may extend from the proximal hub 60, across the proximal face 66 of the expandable framework 52, and over the perimeter shoulder portion 64 to a plurality of side attachment points 74 disposed along a laterally facing surface 69 of the expandable framework 52.

In some embodiments, one or more anchoring element(s) of the plurality of anchoring elements 56 may extend through the occlusive covering 70 at the plurality of side attachment points 74. In some embodiments, the one or more anchoring element(s) of the plurality of anchoring elements 56 extending through the occlusive covering 70 may attach and/or secure the occlusive covering 70 to the expandable framework 52. Other configurations are also contemplated.

In some embodiments, the occlusive covering 70 may be mechanically secured to the proximal face 66 of the expandable framework 52 at a plurality of locations 72. In some embodiments, the occlusive covering 70 may be secured and/or mechanically secured directly to the proximal face 66 of the expandable framework 52 at the plurality of locations 72. In some embodiments, the plurality of locations 72 may be disposed within the distally recessed portion 67 of the proximal face 66. In some embodiments, at least a portion of the occlusive covering 70 may be disposed within the distally recessed portion 67 of the proximal face 66. In some embodiments, the occlusive covering 70 may be secured and/or mechanically secured directly to the proximal face 66 of the expandable framework 52 at the plurality of locations 72 within the distally recessed portion 67 of the proximal face 66. In some embodiments, the plurality of locations 72 may be spaced apart equidistantly around the proximal hub 60. In some embodiments, securing and/or attaching the occlusive covering 70 to the proximal face 66 of the expandable framework 52 at the plurality of locations 72 may hold the occlusive covering 70 closer and/or tighter to the proximal face 66 of the expandable framework 52, thereby reducing the risk of incorrectly diagnosing DRT. When the occlusive covering 70 gets spaced away from the proximal face 66 of the expandable framework 52 after deployment and/or implantation, endothelization across the occlusive covering 70 may appear via CT imaging as DRT instead of the desired endothelization. In some cases, DRT may warrant further intervention. Accordingly, avoiding a misdiagnosed DRT is desirable and may avoid unnecessary procedure(s) and/or intervention(s).

In some embodiments, in the deployed configuration, at least a portion of the occlusive covering 70 extending across the proximal face 66 of the expandable framework 52 may extend distally of the proximal surface 68. In some embodiments, in the deployed configuration, each of the plurality of locations 72 may be disposed distal of the proximal collar 58, the proximal hub 60, the proximal surface 68, and/or the perimeter shoulder portion 64. In some embodiments, in the deployed configuration, the occlusive covering 70 may extend along a path that extends distally from the proximal hub 60 to the plurality of locations 72 and proximally from the plurality of locations 72 toward and/or to the proximal surface 68 and/or the perimeter shoulder portion 64.

In some embodiments, the occlusive covering 70 may be a woven structure, a knitted structure, a fabric structure, a textile structure, and/or a membrane or film having a plurality of apertures formed therein and/or extending therethrough. Additionally, it shall be understood that in some embodiments, a knitted structure, which may include warp knitting, weft knitting, woven knitting, etc., may be preferred, even though such structure is not expressly illustrated. Other configurations are also contemplated.

In some embodiments, the occlusive covering 70 may comprise a plurality of pores (e.g., a plurality of openings, and/or apertures). In some embodiments, the plurality of pores (e.g., a plurality of openings, and/or apertures) may extend through the occlusive covering 70 from a first side to a second side. In some alternative embodiments, the occlusive covering 70 may be non-porous.

In some embodiments, the occlusive covering 70 may be permeable or impermeable to blood and/or other fluids, such as water. In some embodiments, the occlusive covering 70 may be designed, sized, and/or configured to prevent thrombus and/or embolic material from passing out of the left atrial appendage into the left atrium and/or the patient’s bloodstream. In some embodiments, the occlusive covering 70 may promote endothelization after implantation, thereby effectively and/or permanently removing the target site (e.g., the left atrial appendage, etc.) from the patient’s circulatory system. Some suitable, but non-limiting, examples of materials for the occlusive covering 70 are discussed below. In one preferred configuration, the occlusive covering 70 may be formed from a polymeric material.

In some embodiments, the occlusive covering 70 may include a surface treatment configured to promote endothelization on and/or across the occlusive covering 70. In some embodiments, the occlusive covering 70 may include the surface treatment disposed on and/or surrounding a portion of an outer surface and/or a proximally facing surface of the occlusive covering 70. In some embodiments, the occlusive covering 70 may include the surface treatment disposed on and/or surrounding an entire outer surface and/or an entire proximally facing surface of the occlusive covering 70. In some embodiments, the occlusive covering 70 may be elastic and/or stretchable to accommodate changes in shape and/or size of the expandable framework 52 when the expandable framework 52 is shifted toward and/or to the deployed configuration.

In some embodiments, the occlusive covering 70 may be sutured to the proximal face 66 of the expandable framework 52, as seen in FIGS. 3 and 5. In some embodiments, the occlusive covering 70 may be mechanically secured to and/or directly to the proximal face 66 of the expandable framework 52 by a plurality of sutures 76 at the plurality of locations 72. In some embodiments, a single suture may secure the occlusive covering 70 to the proximal face 66 of the expandable framework 52 at each location. In some embodiments, multiple sutures may secure the occlusive covering 70 to the proximal face 66 of the expandable framework 52 at each location. Other configurations, including combinations thereof, are also contemplated.

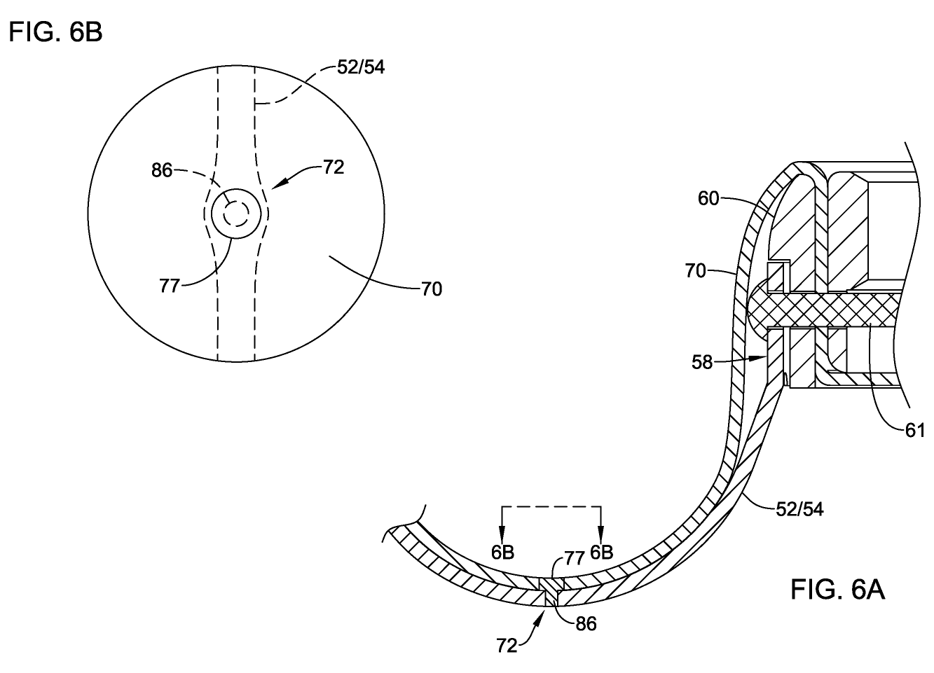

FIGS. 6A and 6B illustrate an alternative configuration of the occlusive implant 50 and the occlusive covering 70 secured thereto. FIG. 6B is a top view of a portion of FIG. 6A taken along line 6B-6B. In some embodiments, the occlusive covering 70 may be mechanically secured to and/or directly to the proximal face 66 of the expandable framework 52 at the plurality of locations 72 with a plurality of melted plugs 77. In some embodiments, the expandable framework 52 and/or the plurality of interconnected struts 54 may comprise apertures 86 formed within individual struts of the plurality of interconnected struts 54 at the plurality of locations 72. For example, an individual strut of the plurality of interconnected struts 54 is illustrated in cross-section in FIG. 6A. In one example, the plurality of melted plugs 77 may be formed from a polymeric material that is compatible with the occlusive covering 70. Other configurations are also contemplated. In some embodiments, plugs of material (e.g., polymeric material) may be inserted through the occlusive covering 70 and into the apertures 86 formed within individual struts of the plurality of interconnected struts 54 at the plurality of locations 72. Subsequently, the plugs of material may be heated and/or melted (e.g., reflowed) to bond with the occlusive covering 70 and/or to completely fill the apertures 86, thereby preventing the plurality of melted plugs 77 from detaching from and/or from disengaging from the expandable framework 52 and/or the plurality of interconnected struts 54. In an alternative configuration, the occlusive covering 70 may be mechanically secured to and/or directly to the proximal face 66 of the expandable framework 52 at the plurality of locations 72 via welding.

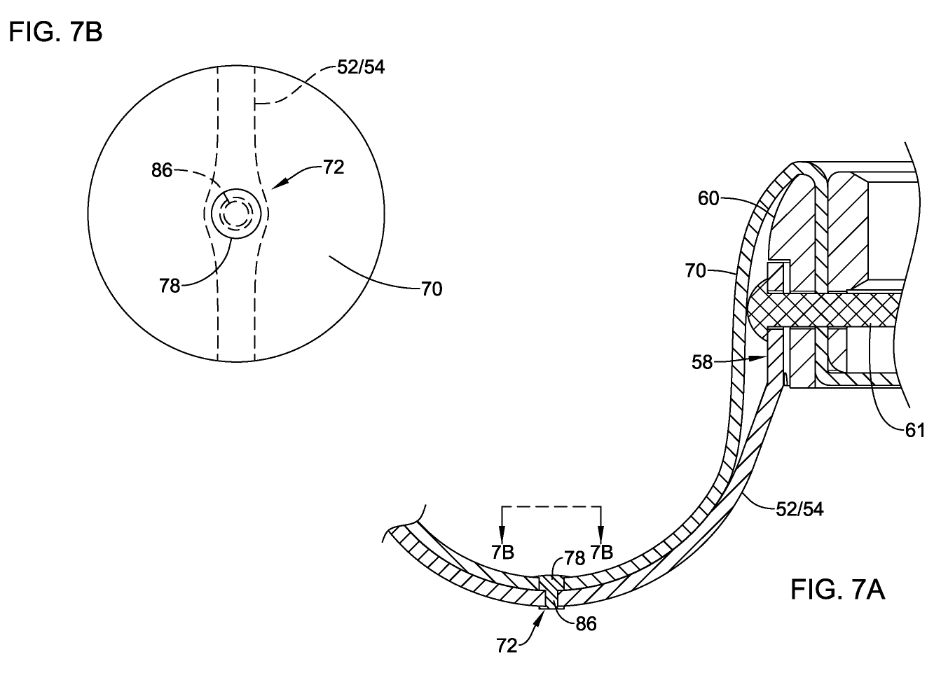

In another alternative configuration, in some embodiments, the occlusive covering 70 may be mechanically secured to and/or directly to the proximal face 66 of the expandable framework 52 at the plurality of locations 72 with a plurality of rivets 78, as seen in FIGS. 7A-7B. FIG. 7B is a top view of a portion of FIG. 7A taken along line 7B-7B. In some embodiments, the plurality of rivets 78 may be inserted through the occlusive covering 70 and into the apertures 86 formed within individual struts of the plurality of interconnected struts 54 at the plurality of locations 72. Subsequently, the plurality of rivets 78 may be deformed using known processes to secure the occlusive covering 70 to the expandable framework 52 and/or the plurality of interconnected struts 54.

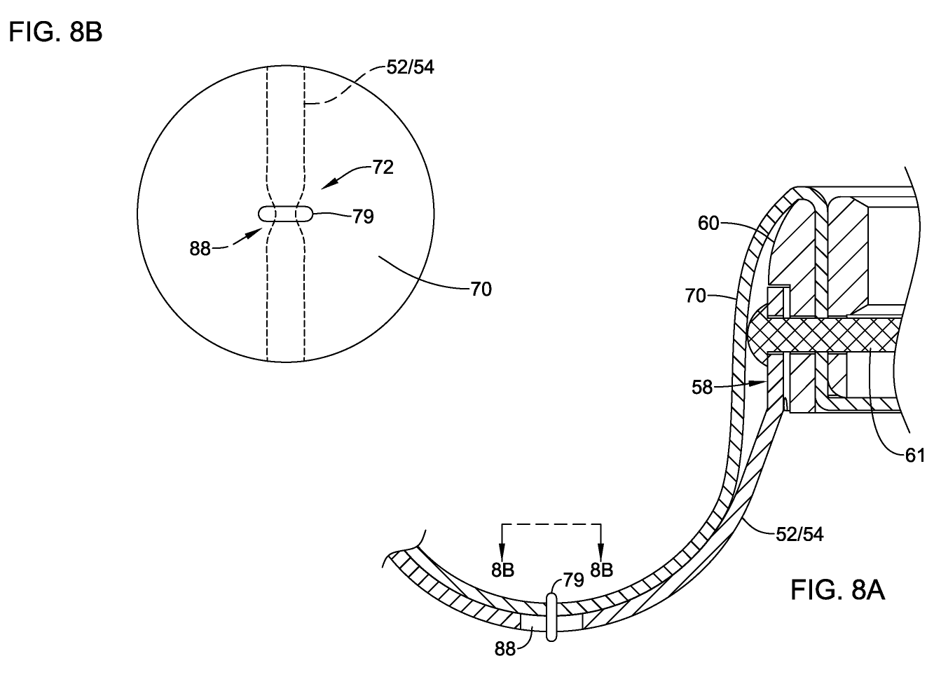

FIGS. 8A and 8B illustrate an alternative configuration of the occlusive implant 50 and the occlusive covering 70 secured thereto. FIG. 8B is a top view of a portion of FIG. 8A taken along line 8B-8B. In some embodiments, the occlusive covering 70 may be mechanically secured to and/or directly to the proximal face 66 of the expandable framework 52 at the plurality of locations 72 with a plurality of staples 79. In some embodiments, the expandable framework 52 and/or the plurality of interconnected struts 54 may comprise lateral notches 88 formed within individual struts of the plurality of interconnected struts 54 at the plurality of locations 72. In some embodiments, the plurality of staples 79 may be inserted through the occlusive covering 70 and around the lateral notches 88 formed within individual struts of the plurality of interconnected struts 54 at the plurality of locations 72 (e.g., straddling the plurality of interconnected struts 54 within the lateral notches 88). Subsequently, the plurality of staples 79 may be crimped around the plurality of interconnected struts 54 at the plurality of locations 72, thereby securing the occlusive covering 70 to the expandable framework 52 and/or the plurality of interconnected struts 54. In at least some embodiments, the plurality of staples 79 may be formed from a metallic material. Other suitable materials are also contemplated.

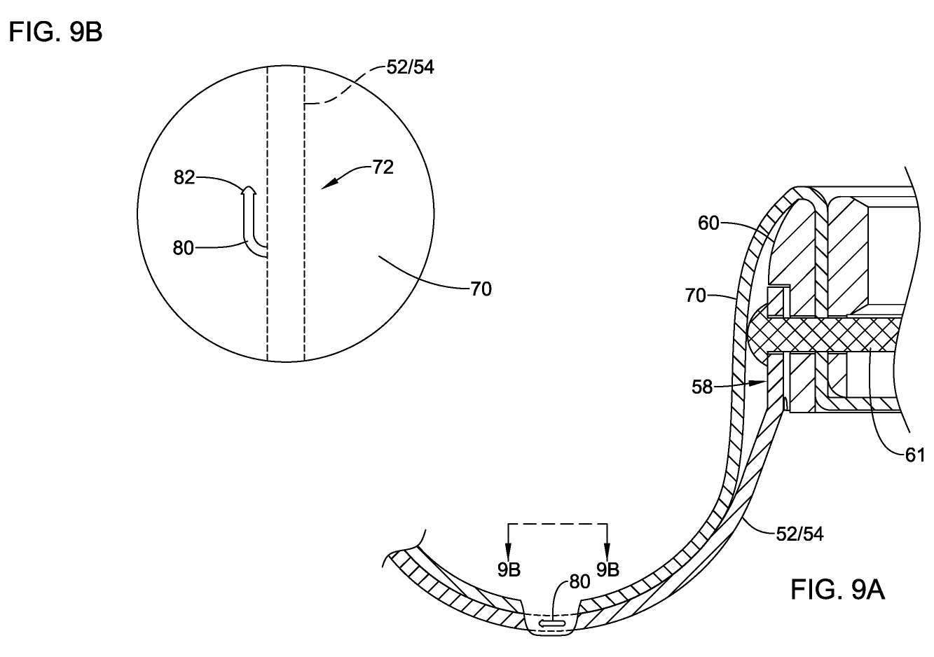

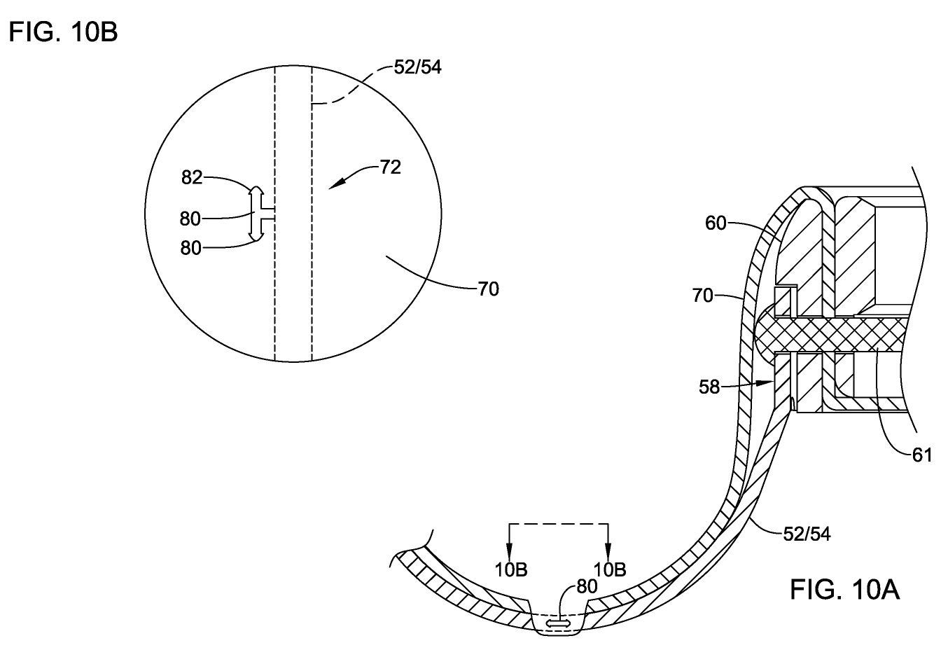

In some embodiments, the occlusive covering 70 may be mechanically secured to and/or directly to the proximal face 66 of the expandable framework 52 at the plurality of locations 72 with a plurality of hooks 80. In at least some embodiments, the plurality of hooks 80 may be monolithically formed with the expandable framework 52 and/or the plurality of interconnected struts 54. In some embodiments, the plurality of hooks 80 may be disposed laterally of their respective attachment points to the plurality of interconnected struts 54. In some alternative embodiments, the plurality of hooks 80 may be disposed proximally of their respective attachment points to the plurality of interconnected struts 54 in the deployed configuration. Other configurations are also contemplated. In some embodiments, each hook of the plurality of hooks 80 may include at least one barb 82 disposed at the free end(s) thereof. In some embodiments, each hook of the plurality of hooks 80 may be single-ended (e.g., may have a single free end), as seen in FIGS. 9A-9B, where FIG. 9A is a cross-sectional view with partial cutaway. FIG. 9B is a top view of a portion of FIG. 9A taken along line 9B-9B. In some embodiments, each hook of the plurality of hooks 80 may be double-ended (e.g., may have two free ends), as seen in FIGS. 10A-10B, where FIG. 10A is a cross-sectional view with partial cutaway. FIG. 10B is a top view of a portion of FIG. 10A taken along line 10B-10B. Other configurations are also contemplated. In some embodiments, the expandable framework 52 may comprise both single-ended and double-ended hooks in various combinations. In some embodiments, the plurality of hooks 80 may be inserted through the occlusive covering 70 at the plurality of locations 72 to secure the occlusive covering 70 to the expandable framework 52 and/or the plurality of interconnected struts 54. In some embodiments, the at least one barb 82 may prevent the occlusive covering 70 from freely slipping off of the plurality of hooks 80. Other configurations are also contemplated.

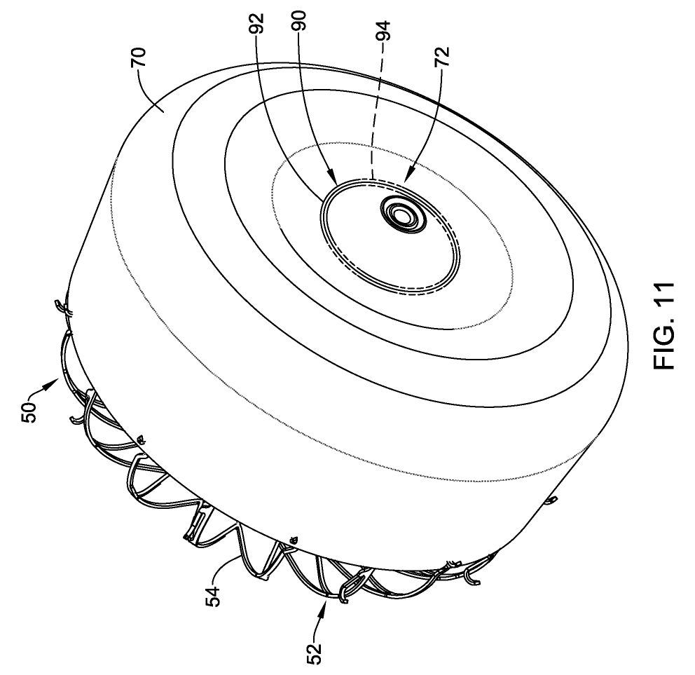

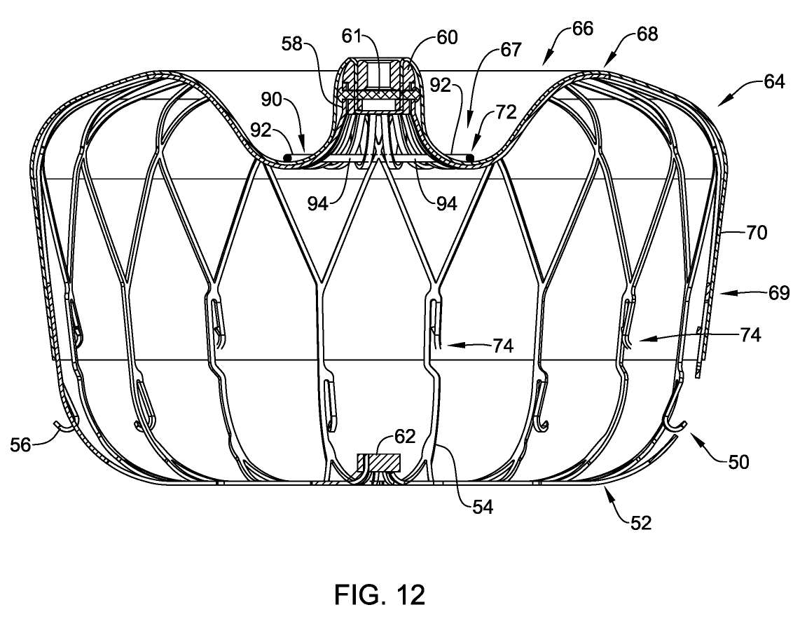

FIGS. 11 and 12 illustrate an alternative configuration of the occlusive implant 50 and the occlusive covering 70 secured thereto. In some embodiments, the occlusive covering 70 may be mechanically secured to and/or directly to the proximal face 66 of the expandable framework 52 with a wire 90 extending circumferentially around the proximal collar 58 and/or the proximal hub 60. In some embodiments, the wire 90 may be interwoven through the occlusive covering 70 and the proximal face 66 of the expandable framework 52. For example, the wire 90 may be interwoven through the occlusive covering 70 and the plurality of interconnected struts 54. In some embodiments, a plurality of first segments 92 of the wire 90 is disposed proximal of the occlusive covering 70 and the proximal face 66 and/or the plurality of interconnected struts 54 of the expandable framework 52, and a plurality of second segments 94 of the wire 90 is disposed distal of the occlusive covering 70 and the proximal face 66 and/or the plurality of interconnected struts 54 of the expandable framework 52. In some embodiments, the plurality of first segments 92 of the wire 90 may be discontinuous with each other. In some embodiments, the plurality of second segments 94 of the wire 90 may be discontinuous with each other. The plurality of first segments 92 and the plurality of second segments 94 may combine end-to-end to form a continuous structure (e.g., the wire 90). In some embodiments, the wire 90 may be fixedly attached directly to the expandable framework 52 and/or the plurality of interconnected struts 54, such as by welding, adhesive bonding, etc., at one end or at both ends. In some embodiments, the wire 90 may be fixedly attached directly to the expandable framework 52 and/or the plurality of interconnected struts 54, such as by welding, adhesive bonding, etc., at at least one medial location along the length of the wire 90. In some embodiments, the wire 90 may be devoid of fixed attachment directly to the expandable framework 52 and/or the plurality of interconnected struts 54. Other configurations, including combinations thereof, are also contemplated. In some embodiments, the wire 90 may be formed from a metallic material. In one preferred configuration, the wire 90 may be formed from nickel-titanium alloy (e.g., nitinol). Some suitable, but non-limiting, examples of materials for the wire 90, including metallic materials, polymeric materials, composite materials, etc., are discussed below. In some embodiments, the wire 90 may be used to identify the location of the occlusive covering 70 post-implantation when evaluating for DRT.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the expandable framework, the occlusive fabric, the wire, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. An occlusive implant system, comprising:
an occlusive implant comprising an expandable framework configured to shift between a collapsed configuration and a deployed configuration, wherein the expandable framework comprises a proximal hub, a perimeter shoulder portion spaced apart radially outward from the proximal hub in the deployed configuration, and a proximal face extending between the proximal hub and the perimeter shoulder portion; and
an occlusive covering secured to the expandable framework;
wherein the occlusive covering extends from the proximal hub, across the proximal face of the expandable framework, and over the perimeter shoulder portion;
wherein the occlusive covering is mechanically secured to the proximal face of the expandable framework at a plurality of locations.

2. The occlusive implant system of claim 1, wherein the proximal face of the expandable framework includes a distally recessed portion surrounding the proximal hub in the deployed configuration.

3. The occlusive implant system of claim 2, wherein the proximal face of the expandable framework includes a proximal surface disposed radially outward of the distally recessed portion and proximal of the distally recessed portion in the deployed configuration.

4. The occlusive implant system of claim 3, wherein at least a portion of the occlusive covering extending across the proximal face of the expandable framework extends distally of the proximal surface.

5. The occlusive implant system of claim 2, wherein at least a portion of the occlusive covering is disposed within the distally recessed portion.

6. The occlusive implant system of claim 2, wherein the plurality of locations is disposed within the distally recessed portion.

7. The occlusive implant system of claim 1, wherein the occlusive covering is sutured to the proximal face of the expandable framework.

8. The occlusive implant system of claim 1, wherein the occlusive covering is mechanically secured to the proximal face of the expandable framework with a plurality of melted plugs.

9. The occlusive implant system of claim 1, wherein the occlusive covering is mechanically secured to the proximal face of the expandable framework with a plurality of hooks monolithically formed with the expandable framework.

10. The occlusive implant system of claim 1, wherein the occlusive covering is mechanically secured to the proximal face of the expandable framework with a wire extending circumferentially around the proximal hub.

11. The occlusive implant system of claim 10, wherein the wire is interwoven through the occlusive covering and the proximal face of the expandable framework.

12. The occlusive implant system of claim 10, wherein a plurality of first segments of the wire is disposed proximal of the occlusive covering and a plurality of second segments of the wire is disposed distal of the proximal face of the expandable framework.

13. An occlusive implant system, comprising:
an occlusive implant comprising an expandable framework configured to shift between a collapsed configuration and a deployed configuration, wherein the expandable framework comprises a proximal hub, a perimeter shoulder portion spaced apart radially outward from the proximal hub in the deployed configuration, and a proximal face extending from the proximal hub to the perimeter shoulder portion; and
an occlusive covering secured to the proximal hub and the expandable framework;
wherein the occlusive covering extends from the proximal hub, across the proximal face of the expandable framework, and over the perimeter shoulder portion to a plurality of side attachment points disposed along a laterally facing surface of the expandable framework;
wherein the occlusive covering is secured directly to the proximal face of the expandable framework at a plurality of locations.

14. The occlusive implant system of claim 13, wherein the expandable framework comprises a plurality of interconnected struts.

15. The occlusive implant system of claim 14, wherein the occlusive covering is secured directly to the plurality of interconnected struts at the plurality of locations within a distally recessed portion of the proximal face.

16. The occlusive implant system of claim 14, wherein the plurality of interconnected struts comprises apertures formed within individual struts of the plurality of interconnected struts at the plurality of locations.

17. The occlusive implant system of claim 14, wherein the plurality of interconnected struts comprises lateral notches formed within individual struts of the plurality of interconnected struts at the plurality of locations.

18. The occlusive implant system of claim 13, wherein in the deployed configuration, the occlusive covering extends along a path that extends distally from the proximal hub to the plurality of locations and proximally from the plurality of locations toward the perimeter shoulder portion.

19. The occlusive implant system of claim 13, wherein the plurality of locations is circumferentially spaced apart equidistantly around the proximal hub.

20. An occlusive implant system, comprising:
an occlusive implant comprising an expandable framework configured to shift between a collapsed configuration and a deployed configuration, wherein the expandable framework comprises a proximal hub, a perimeter shoulder portion spaced apart radially outward from the proximal hub in the deployed configuration, and a proximal face extending between the proximal hub and the perimeter shoulder portion; and
an occlusive covering extending radially outward from the proximal hub across the proximal face of the expandable framework;
wherein the occlusive covering is mechanically secured to the proximal face of the expandable framework at a plurality of locations;
wherein in the deployed configuration, each of the plurality of locations is disposed distal of the perimeter shoulder portion.
